(19) United States
(12) Patent Application Publication
(10) Pub. No.: US 2012/0022310 A1
(43) Pub. Date: Jan. 26, 2012

(54) PROCESS FOR PREPARING AROMATICS FROM METHANE

(75) Inventors: Christian SCHNEIDER, Mannheim (DE); Sebastian Ahrens, Wiesloch (DE); Kati Bachmann, Mannheim (DE); Joana Coelho Tsou, Bruessel (BE); Thomas Heidemann, Viernheim (DE); Annebart Engbert Wentink, Mannheim (DE)

(73) Assignee: BASF SE, Ludwigshafen (DE)

(21) Appl. No.: 13/186,592

(22) Filed: Jul. 20, 2011

Related U.S. Application Data
(60) Provisional application No. 61/366,172, filed on Jul. 21, 2010.

(51) Int. Cl. C07C 2/00

(52) U.S. Cl. 585/415

(57) ABSTRACT

The present invention relates to a process for carrying out endothermic, heterogeneously catalyzed reactions in which the reaction of the starting materials is carried out in the presence of a mixture of inert heat transfer particles and catalyst particles, where the catalyst particles are regenerated in a nonoxidative atmosphere at regular intervals and the heat of reaction required is introduced by separating off the inert heat transfer particles, heating the heat transfer particles in a heating zone and recirculating the heated heat transfer particles to the reaction zone. The process of the invention is particularly suitable for the nonoxidative dehydroaromatization of C₁-C₄-aliphatics in the presence of zeolite-comprising catalysts.

This patent application claims the benefit of pending U.S. provisional patent application Ser. No. 61/366,172 filed on Jul. 21, 2010, incorporated in its entirety herein by reference.

### BACKGROUND OF THE INVENTION

The present invention relates to a process for carrying out endothermic, heterogeneously catalyzed reactions in which the reaction of the starting materials is carried out in the presence of a mixture of inert heat transfer particles and catalyst particles, where the catalyst particles are regenerated in a nonoxidative atmosphere at regular intervals and the heat of reaction required is introduced by separating off the inert heat transfer particles, heating the heat transfer particles in a heating zone and recirculating the heated heat transfer particles to the reaction zone. The process of the invention is particularly suitable for the nonoxidative dehydroaromatization of C₁-C₄-aliphatics in the presence of zeolite-comprising catalysts.

In many endothermic reactions, supplying the energy required for the reactions presents a particular challenge. If the reaction is indirectly heated, large heat exchange surfaces are necessary and make the process complicated in terms of apparatus and expensive. In addition, undesirable secondary reactions, for example carbonization in the reaction of hydrocarbons, frequently take place on the heat transfer surfaces because of the relatively high temperatures. This also applies, inter alia, to the nonoxidative dehydroaromatization of methane (DHAM), which is an endothermic reaction and requires supply of heat from the outside.

One possible way of directly introducing heat of reaction is the use of particles which do not participate in the reaction as heat transfer particles which are heated in an external reactor, optionally together with catalyst particles, by direct contact with combustion offgases or by direct combustion of a fuel to a temperature above the reaction temperature and are subsequently returned to the reaction zone. The energy required for the reaction is subsequently transferred by direct contact of the inert heat transfer particles with the catalyst particles. Processes of this type in which inert particles are used for introducing the heat of reaction are known from the prior art.

A further problem encountered in many reactions catalyzed by solids is increasing deactivation of the catalyst used and the latter has to be regenerated regularly. Thus, in the industrial implementation of dehydroaromatization of C₁-C₄-aliphatics under nonoxidative conditions, carbonization of the catalysts occurs and reduces the activity of the catalyst in a relatively short time, leading to short production cycles and an increased need for regeneration. The carbonization is frequently associated with a shortened life of the catalyst. Regeneration of the catalyst is not unproblematical since, firstly, the initial activities have to be restored regularly and, secondly, this has to be possible after a large number of cycles in order to achieve an economical process.

The carbonaceous deposits also have an adverse effect on the mass balance or the yield, since each molecule which is converted into carbonaceous deposit is no longer available for the desired reaction to form aromatics. The carbonaceous deposit selectivities achieved hitherto in the prior art are in most cases over 20% based on the aliphatics reacted.

Processes in which the heat of reaction is supplied by heating heat transfer particles and the catalyst particles have to be regenerated regularly are known.

U.S. Pat. No. 5,030,338 describes a process for aromatizing aliphatics in the presence of zeolites and catalysts and inert particles, in which the mixture of deactivated catalyst and inert particles is taken off from the reaction zone, the mixture is freed of adhering hydrocarbons by stripping and the stripped mixture is separated into a stream comprising predominantly catalyst particles and a second stream comprising essentially inert particles. The stream comprising predominantly catalyst is transferred to a regeneration zone and regenerated by means of an oxygen-comprising gas. The second stream which comprises predominantly the inert particles is introduced into a combustion zone; in this combustion zone, the inert particles are heated by combustion of a fuel in oxygen. The heat of reaction is introduced into the reaction zone by means of the mixture of catalyst particles and inert particles.

U.S. Pat. No. 2,763,596 relates to a process for the treatment of hydrocarbons in the presence of hydrogen and in the presence of solid catalyst particles, with the aromaticity of the hydrocarbons being increased. To introduce the required heat of reaction into the reaction zone, heat transfer particles are circulated firstly between the regeneration zone and the reaction zone and secondly between the reaction zone and the heating zone. In the regeneration zone, inert particles and catalyst particles are regenerated by freeing them of carbon deposits by means of oxygen to liberate heat; in the heating zone, the inert particles are heated in combustion offgases.

In the processes known from the prior art, the catalyst particles and the inert heat transfer particles are subjected to severe mechanical, chemical and thermal stresses due to the many transport operations necessary between reaction zone, regeneration zone and heating zone and these lead to a shortening of the life of the catalysts.

There is therefore a need for further, improved processes beyond those known from the prior art for carrying out endothermic reactions which are heterogeneously catalyzed by catalysts, especially reactions catalyzed by zeolite-comprising catalysts.

### BRIEF SUMMARY OF THE INVENTION

This object is achieved according to the invention by a process for carrying out endothermic, heterogeneously catalyzed reactions, which comprises the steps
(a) carrying out the reaction in a reaction zone in the presence of a mixture comprising catalyst particles and inert heat transfer particles,
(b) regeneration of the catalyst particles, comprising
   (b1) transfer of the mixture comprising catalyst particles and optionally inert heat transfer particles into a regeneration zone,
   (b2) regeneration of the catalyst particles and optionally the inert heat transfer particles in a nonoxidative atmosphere and
   (b3) recirculation of the regenerated catalyst particles to the reaction zone and
(c) introduction of heat into the reaction zone, which comprises the steps
   (c1) separation of the inert heat transfer particles from the catalyst particles between step (a) and (b), during step (b) or after step (b),
   (c2) transfer of the inert heat transfer particles which have been separated off into a heating zone and
   (c3) heating of the inert heat transfer particles and recirculation of the heated inert heat transfer particles to the reaction zone.

In a preferred embodiment, the reaction in step (a) is the nonoxidative dehydroaromatization of C₁-C₄-aliphatics in the presence of zeolite-comprising catalyst particles.

In a further preferred embodiment, the catalyst particles and optionally the inert heat transfer particles are regenerated in step (b2) by introduction of a hydrogen-comprising regeneration gas stream.

It has surprisingly been found that the separation of the catalyst particles from the inert heat transfer particles before heating of the inert heat transfer particles in a riser or in hot combustion offgases and regeneration of the catalyst particles in a nonoxidative atmosphere increases the life of the catalyst. As has been discovered by the inventors, the reactivity of the zeolite-comprising catalyst decreases, for example, in the DHAM in the presence of even small amounts of water at the temperatures of more than 700° C. which usually prevail during heating, see Example 1. This is associated with a decrease in the degree of crystallinity of the zeolite comprised in the catalyst. The combustion of fuels comprising hydrogen atoms, e.g. methane, with oxygen or air forms water vapor which irreversibly damages the zeolite comprised in the catalyst during heating of the catalyst particles by means of the combustion or the combustion offgases formed. Introduction of the heat of reaction for the dehydroaromatization of methane by external heating of the catalyst particles by combustion of a fuel such as methane in a riser directly in the presence of the particles to be heated, as described, for example, in US 2008/0249342 A1, can therefore irreversibly damage the catalyst. This problem could be overcome by not heating the catalyst by means of direct contact with the combustion offgases but instead heating a water-free gas stream (for example nitrogen or hydrogen) by means of the combustion offgases and then heating the catalyst by direct contact with this gas stream. However, this process variant is technically complicated (heat transfer area, inert gas circuit) and costly. In addition, the total energy consumption in this process variant is higher than in the case of direct heating because of engineering limitations, e.g. the inert gas blower used.

The process of the invention has the advantage that the catalyst particles do not come into direct contact with the combustion offgases and are therefore not damaged by the water present therein. Since the catalyst particles circulate only between regeneration zone and reaction zone (and are not also conveyed into a heating zone) and the inert heat transfer particles either go together with the catalyst particles into the regeneration zone or are separated off beforehand and transferred into the heating zone, the transport distances required are significantly shorter than in the processes of the prior art. This has a favorable effect on the operating life of the catalyst.

In the case of reactions in which the catalyst particles are deactivated by deposition of carbonaceous material and/or carbon-comprising deposits, regeneration by introduction of a hydrogen-comprising regeneration gas stream is particularly advantageous since in this case the carbon comprised in the deposits can be converted back into methane and utilized further, particularly when methane is used as starting material in the reaction in step (a). If the inert heat transferrers are separated off from the catalyst particles in the regeneration zone, carbon-comprising deposits present on the inert heat transfer particles may also be converted back into methane when a hydrogen-comprising regeneration gas stream is used.

Very particular preference is given to an embodiment of the present invention in which the heat transfer particles and the catalyst particles are separated from one another in the regeneration zone or later and the regeneration is carried out by means of a hydrogen-comprising regeneration gas. In this case, the carbon-comprising deposits on both types of particles can be converted into hydrocarbons. If the regeneration zone directly follows the reaction zone, the transport distances for the catalyst particles become very short and the mechanical stress on the catalyst particles used is reduced further.

### BRIEF DESCRIPTION OF THE SEVERAL VIEWS OF THE DRAWINGS

FIG. 1a is a cross-sectional illustration of a reactor for carrying out a nonoxidative aromatization of methane with regeneration of a deactivated catalyst by means of a hydrogen-comprising regeneration gas stream.

FIG. 1b is is a cross-sectional illustration of a reactor for carrying out a nonoxidative aromatization of methane with regeneration of a deactivated catalyst by means of a hydrogen-comprising regeneration gas stream.

FIG. 1c is is a cross-sectional illustration of a reactor for carrying out a nonoxidative aromatization of methane with regeneration of a deactivated catalyst by means of a hydrogen-comprising regeneration gas stream.

FIG. 1d is is a cross-sectional illustration of a reactor for carrying out a nonoxidative aromatization of methane with regeneration of a deactivated catalyst by means of a hydrogen-comprising regeneration gas stream.

FIG. 2 shows the benzene selectivity and the selectivity for carbonaceous material as a function of the reaction time.

### DETAILED DESCRIPTION OF THE INVENTION

For the purposes of the invention, “heterogeneously catalyzed” means that at least part of the catalyst or catalysts used, preferably the total amount of the catalyst or catalysts used, is present as a solid and the starting material or starting materials used are present in gaseous and/or liquid form.

“Inert heat transfer particles” in the present case are particles which do not have an adverse effect on the reaction in step (a), preferably do not participate in the reaction carried out in step (a) and serve essentially as medium for introducing heat from the outside into the reaction zone.

In the context of regeneration, nonoxidative means, for the purposes of the present invention, that the carbonaceous deposits on the catalyst which originate from the reaction in step (a) are, for the purpose of regenerating the catalyst, not converted by means of oxidants such as air or oxygen into CO and/or CO₂ but are instead removed reductively. In particular, the concentration of oxidants in the mixture used for the regeneration in step (b2) is below 5% by weight, preferably below 1% by weight, particularly preferably below 0.1% by weight, very particularly preferably free of oxidants.

For the purposes of the present invention, nonoxidative means, in the context of the dehydroaromatization (DHAM) of C₁-C₄-aliphatics, that the concentration of oxidants such as oxygen or nitrogen oxides in the feed stream E is below 5% by weight, preferably below 1% by weight, particularly preferably below 0.1% by weight. The mixture is very particularly preferably free of oxygen. Particular preference is likewise given to a concentration of oxidants in the mixture E which is the same as or lower than the concentration of oxidants in the source from which the C₁-C₄-aliphatics originate.

In step (a) of the process of the invention, an endothermic, heterogeneously catalyzed reaction is carried out in the presence of a catalyst, preferably a zeolite-comprising catalyst. This reaction can in principle be any endothermic, heterogeneously catalyzed reaction in which the required heat of reaction is to be introduced directly into the reaction zone and the catalyst particles have to be regenerated regularly. Such reactions are, for example, dehydrogenations, in particular the nonoxidative dehydroaromatization of aliphatics, the dehydrogenative aromatization of cycloaliphatics and also gasification reactions and pyrolyses.

According to the invention, the reaction carried out in step (a) is preferably the nonoxidative dehydroaromatization of C₁-C₄-aliphatics. This is described in detail below.

In the nonoxidative dehydroaromatization of C₁-C₄-aliphatics (DHAM), a feed stream E comprising at least one aliphatic having from 1 to 4 carbon atoms is reacted in the presence of at least one catalyst to liberate hydrogen and form aromatics. These aliphatics include, for example, methane, ethane, propane, n-butane, i-butane, ethene, propene, 1- and 2-butene, isobutene. In one embodiment of the invention, the feed stream E comprises at least 50 mol %, preferably at least 60 mol %, particularly preferably at least 70 mol %, very preferably at least 80 mol %, in particular at least 90 mol %, of C₁-C₄-aliphatics.

Among the aliphatics, particular preference is given to using the saturated alkanes, and feed stream E then comprises at least 50 mol %, preferably at least 60 mol %, particularly preferably at least 70 mol %, very preferably at least 80 mol %, in particular at least 90 mol %, of alkanes having from 1 to 4 carbon atoms.

Among the alkanes, methane and ethane are preferred, in particular methane. In this embodiment of the present invention, the feed stream E comprises at least 50 mol %, preferably at least 60 mol %, particularly preferably at least 70 mol %, very preferably at least 80 mol %, in particular at least 90 mol %, of methane.

Natural gas is preferably used as source of the C₁-C₄-aliphatics. The typical composition of natural gas is as follows: from 75 to 99 mol % of methane, from 0.01 to 15 mol % of ethane, from 0.01 to 10 mol % of propane, up to 6 mol % of butane, up to 30 mol % of carbon dioxide, up to 30 mol % of hydrogen sulfide, up to 15 mol % of nitrogen and up to 5 mol % of helium. The natural gas can be purified and concentrated by methods known to those skilled in the art before use in the process of the invention. Purification includes, for example, the removal of any hydrogen sulfide or carbon dioxide and further compounds which are undesirable in the subsequent process which may be present in the natural gas.

The C₁-C₄-aliphatics comprised in the feed stream E can also originate from other sources, for example have been obtained in oil refining. The C₁-C₄-aliphatics can also have been produced regeneratively (e.g. biogas) or synthetically (e.g. Fischer-Tropsch synthesis).

If biogas is used as source of C₁-C₄-aliphatics, the feed stream E can additionally comprise ammonia, traces of lower alcohols and further components typical of biogas.

In a further embodiment of the process of the invention, LPG (liquid petroleum gas) can be used as feed stream E. In a further embodiment of the process of the invention, LNG (liquefied natural gas) can be used as feed stream E.

Hydrogen, carbon monoxide, carbon dioxide, nitrogen and also one or more noble gases can additionally be mixed into the feed stream E. The feed stream E preferably comprises hydrogen, more preferably from 0.1 to 10% by volume of hydrogen, particularly preferably from 0.1 to 5% by volume of hydrogen.

In the reaction zone, the feed stream E is reacted under nonoxidative conditions in the presence of a particulate catalyst to form a product stream P comprising aromatic hydrocarbons. In the dehydroaromatization, the C₁-C₄-aliphatics comprised in the feed stream E are dehydrogenated and cyclized to form the corresponding aromatics, with hydrogen being liberated. The DHAM is usually carried out in the presence of suitable catalysts. Such catalysts and processes for producing them are known to those skilled in the art. The DHAM catalysts usually comprise a porous support and at least one metal applied thereto. A crystalline or amorphous inorganic compound is usually used as support.

Preference is given, according to the invention, to the catalyst comprising at least one zeolite as support. Very particular preference is given according to the invention to the zeolite comprised in the catalysts having a structure selected from among the structure types pentasil and MWW and particularly preferably selected from among the structure types MFI, MEL and mixed structures of MFI and MEL and MWW. Very particular preference is given to using a zeolite of the ZSM-5 or MCM-22 type. The naming of the structure types of the zeolites corresponds to those given in W. M. Meier, D. H. Olson and Ch. Baerlocher, “Atlas of Zeolite Structure Types”, Elsevier, 3rd edition, Amsterdam 2001. The synthesis of the zeolites is known to those skilled in the art and can be carried out, for example, starting from alkali metal aluminate, alkali metal silicate and amorphous SiO₂ under hydrothermal conditions. Here, the type of catalyst systems formed in the zeolite can be controlled by means of organic template molecules, via the temperature and further experimental parameters.

The zeolites can comprise further elements such as Ga, B, Fe or In in addition to Al.

The DHAM catalyst usually comprises at least one metal. The metal is usually selected from groups 3 to 12 of the Periodic Table of the Elements (IUPAC). According to the invention, the DHAM catalyst preferably comprises at least one element selected from among the transition metals of main groups 6 to 11. The DHAM catalyst particularly preferably comprises Mo, W, Mn, Tc, Re, Fe, Ru, Os, Co, Rh, Ir, Ni, Pd, Pt, Cu, Ag, Au. In particular, the DHAM catalyst comprises at least one element selected from the group consisting of Mo, W, Re, Fe, Ru, Os, Co, Rh, Ir, Ni, Pd, Pt, Cu. The DHAM catalyst very particularly preferably comprises at least one element selected from the group consisting of Mo, W and Re.

Preference is likewise given, according to the invention, to the DHAM catalyst comprising at least one metal as active component and at least one further metal as dopant. The active component is, according to the invention, selected from among Mo, W, Re, Ru, Os, Rh, Ir, Pd, Pt. The dopant is, according to the invention, selected from the group consisting of Cr, Mn, Fe, Co, Ni, Cu, V, Zn, Zr and Ga, preferably from the group consisting of Fe, Co, Ni, Cu. According to the invention, the DHAM catalyst can comprise more than one metal as active component and more than one metal as dopant. These are in each case selected from among the metals indicated for the active component and the dopant.

The at least one metal is, according to the invention, applied wet-chemically or dry-chemically to the support by methods known to those skilled in the art.

According to the invention, the catalyst comprises from 0.1 to 20% by weight, preferably from 0.2 to 15% by weight, particularly preferably from 0.5 to 10% by weight, in each case based on the total weight of the catalyst, of the at least one metal.

According to the invention, the catalyst can comprise at least one metal from the group of active components in combination with at least one metal selected from the group of dopants. In this case, the concentration of the active component is from 0.1 to 20% by weight, preferably from 0.2 to 15% by weight, particularly preferably from 0.5 to 10% by weight, in each case based on the total weight of the catalyst.

According to the invention, the dopant is in this case present in the catalyst in a concentration of at least 0.1% by weight, preferably at least 0.2% by weight, very particularly preferably at least 0.5% by weight, based on the total weight of the catalyst.

In a further preferred embodiment of the present invention, the catalyst is mixed with a binder. Suitable binders are the customary binders such as binders comprising aluminum oxide and/or Si which are known to those skilled in the art. Particular preference is given to Si-comprising binders; particularly suitable binders of this type are tetraalkoxysilanes, polysiloxanes and colloidal SiO₂ sols or mixtures of the substances mentioned.

If the catalyst according to the invention comprises a binder or mixture of binders, this binder/binder mixture is present in a concentration of from 5 to 80% by weight, based on the total weight of the catalyst, preferably from 10 to 50% by weight, particularly preferably from 10 to 30% by weight.

According to the invention, addition of the binder is followed by a shaping step in which the catalyst composition can be processed by methods known to those skilled in the art to produce shaped bodies. Shaping processes which may be mentioned are, for example, spraying of a suspension comprising the support or the catalyst composition, spray drying, tableting, pressing in the moist or dry state and extrusion. It is also possible to combine two or more of these methods. Auxiliaries such as pore formers and pasting agents or other additives known to those skilled in the art can be used for the shaping step.

Pore formers and/or pasting agents are preferably removed from the shaped body obtained after shaping by means of at least one suitable drying and/or calcination step. The conditions required for this purpose can be selected in a manner analogous to the parameters described above for calcination and are known to those skilled in the art.

The geometry of the catalysts which can be obtained according to the invention can be, for example, spherical (hollow or solid), cylindrical (hollow or solid), ring-, saddle-, star-, honeycomb- or pellet-shaped. Furthermore, extrudates are also possible, for example in rod, trilobe, quatrolobe, star or hollow-cylindrical form. Furthermore, the catalyst composition to be shaped can be extruded, calcined and the extrudates obtained in this way can be broken up and processed to produce crushed material or powder. The crushed material can be separated into various sieve fractions. In a preferred embodiment of the invention, the catalyst is used as spray-dried particles, preferably spray-dried powders. Such particles are preferably round particles. The catalyst particles preferably have a size of from 10 to 200 microns.

Preference is given to using catalyst geometries as are known from the FCC (fluid catalytic cracking) process.

It can be advantageous to activate the catalyst used for the dehydroaromatization of C₁-C₄-aliphatics before the actual reaction.

This activation can be effected by means of a C₁-C₄-alkane such as methane, ethane, propane, butane or a mixture thereof, preferably butane. The activation is carried out at a temperature of from 250 to 850° C., preferably from 350 to 650° C., and a pressure of from 0.5 to 5 bar, preferably from 0.5 to 4 bar. The GHSV (gas hourly space velocity) in the activation is usually from 100 to 4000 h⁻¹, preferably from 500 to 2000 h⁻¹.

However, it is also possible to carry out an activation by the feed stream E per se already comprising the C₁-C₄-alkane or a mixture thereof or the C₁-C₄-alkane or a mixture thereof being added to the feed stream E. The activation is carried out at a temperature of from 250 to 650° C., preferably from 350 to 550° C., and a pressure of from 0.5 to 5 bar, preferably from 0.5 to 2 bar.

In a further embodiment, it is also possible to add hydrogen in addition to the C₁-C₄-alkane.

In a particular embodiment of the present invention, the catalyst is activated by means of an H₂-comprising gas stream which can additionally comprise inert gases such as N₂, He, Ne and Ar.

According to the invention, the dehydroaromatization of C₁-C₄-aliphatics is carried out at temperatures of from 400 to 1000° C., preferably from 500 to 900° C., particularly preferably from 600 to 800° C., in particular from 700 to 800° C., at a pressure of from 0.5 to 100 bar, preferably from 1 to 30 bar, particularly preferably from 1 to 10 bar, in particular from 1 to 5 bar. According to the present invention, the reaction is carried out at a GHSV (gas hourly space velocity, volume flow of starting material/volume of the catalyst bed) of from 10 to 10 000 h⁻¹, preferably from 20 to 3000 h⁻¹.

The C₁-C₄-aliphatics are converted into aromatics with liberation of H₂. The resulting product stream P therefore comprises at least one aromatic hydrocarbon selected from the group consisting of benzene, toluene, ethylbenzene, styrene, xylene and naphthalene. It particularly preferably comprises benzene and toluene. Furthermore, the product stream comprises unreacted C₁-C₄-aliphatics, hydrogen formed and the inert gases comprised in the feed stream E, e.g. N₂, He, Ne, Ar, materials added to the feed stream E, e.g. H₂, and impurities originally present in E.

According to the invention, the heat of reaction required is introduced into the reaction zone by means of inert heat transfer particles. The inert heat transfer particles to be used according to the invention should have a low abrasiveness so that they cause as little damage as possible to the reactor and the transport pipes. The particles should be abrasion-resistant in order to be able to go through very many heat transfer cycles. Furthermore, the inert heat transfer particles must not be too brittle in order to survive the impacts with one another and with the walls of the reactor or pipes undamaged. In addition, they must not have an adverse effect on the reaction carried out in step (a).

In principle, the inert heat transfer particles can be made of all materials from which particles having the abovementioned properties can be made. The heat transfer particles preferably have a rounded shape and particularly preferably have an essentially spherical shape. The material for the inert heat transfer particles can be selected, for example, from the group consisting of glass, ceramic, silicon carbide, metal oxides such as aluminum oxide and mixed oxides of silicon dioxide with aluminum oxide, silicon dioxide with magnesium dioxide, silicon dioxide with thorium dioxide, silicon dioxide with aluminum oxide and zirconium oxide, zirconium oxide, steatite and sand, in particular steatite, preferably from the group consisting of glass spheres, ceramic spheres, silicon carbide particles, Al₂O₃ particles, steatite particles and sand. The inert heat transfer particles are particularly preferably rounded steatite particles, in particular steatite spheres.

The weight ratio of heat transfer particles to catalyst particles in the reaction zone is usually from 2:1 to 1:10, preferably from 1:1 to 1:6. The precise weight ratio depends on the fluidization conditions in the reaction zone and segregation zone and also the properties of the gas and the particles.

The mass flow of the heat transfer particles is determined by the quantity of heat which has to be introduced into the reaction zone in order to compensate for the endothermic nature of the reaction in step (a). The greater the quantity of heat required, the higher the mass flow of heat transfer particles or the temperature thereof. The quantity of heat which may be evolved in the regeneration of the catalyst particles can also play a role. In the case of the DHAM of C₁-C₄-aliphatics, the weight ratio of heat transfer particles to catalyst particles in the reaction zone is usually from 2:1 to 1:10, preferably from 1:1 to 1:6.

In step (b), the catalyst particles are regenerated. For this purpose, the mixture comprising catalyst particles and optionally inert heat transfer particles is, in step (b1), transferred from the reaction zone into a regeneration zone. In the regeneration zone, the catalyst particles and optionally the inert heat transfer particles are regenerated in a nonoxidative atmosphere (step (b2)). According to the invention, the regeneration is preferably carried out by introduction of a hydrogen-comprising regeneration gas stream. If the catalyst has been deactivated in step (a) by deposition of carbonaceous material and further carbon-comprising compounds, these deposits are converted in the regeneration into methane by means of the hydrogen comprised in the regeneration gas stream. The same occurs at least partially in the case of the deposits on the inert heat transfer particles if these have not been separated off from the catalyst particles before the regeneration. The conversion of carbonaceous deposits into methane is exothermic and the catalyst particles and any inert heat transfer particles and also the resulting methane-comprising gas stream M can take up this heat.

The regeneration in step (b2) is usually carried out at temperatures of from 600° C. to 1000° C. and preferably from 700° C. to 900° C. The pressures in the regeneration are usually from 1 bar to 30 bar, preferably from 1 bar to 15 bar and particularly preferably from 1 bar to 10 bar. The regeneration of carbon-comprising deposits and carbonaceous material by means of a hydrogen-comprising regeneration gas stream results in formation of a methane-comprising stream M which, in addition to the methane formed, comprises further compounds formed in the regeneration, unreacted hydrogen and materials originally present in the hydrogen-comprising regeneration gas stream.

The concentration of hydrogen in the regeneration gas stream is usually from 20 to 100% by volume, preferably from 60 to 100% by volume.

The concentration of hydrogen in the regeneration gas stream is preferably calculated so that the methane-comprising gas stream M formed from carbon-comprising deposits in the regeneration preferably comprises not more than 60% by volume, particularly preferably not more than 20% by volume, of hydrogen and very particularly preferably only the amount of hydrogen which corresponds to the thermodynamic equilibrium under these conditions, i.e. the hydrogen introduced has been very largely and preferably completely consumed in the regeneration in step (b2).

If a reaction in which methane can be used as starting material, in particular the DHAM, is carried out in step (a), at least part of the methane formed in the regeneration is, in a particularly preferred embodiment of the invention, fed into the reaction zone in step (a). Particular preference is given to at least 50%, more preferably at least 80%, very particularly preferably at least 90%, of the gas stream M and in particular the entire gas stream M from the regeneration zone being transferred into the reaction zone. The abovementioned percentages are based on the volume of the gas stream M.

In a preferred embodiment of the present invention, the regeneration zone directly adjoins the reaction zone. The transition region between the reaction zone and the regeneration zone is preferably not more than 25%, particularly preferably not more than 10% and very particularly preferably not more than 5%, of the length of the reaction zone. For the present purposes, the length of the reaction zone is the dimension of the reactor parallel to the main flow direction of the gas.

The regeneration zone is particularly preferably arranged below the reaction zone and has not more than the same cross section perpendicular to the main flow direction of the particles as the reaction zone, preferably a cross section which is at least 20% smaller.

The regeneration zone can be adjoined by a stripping zone. In the stripping zone, the inert heat transfer particles are freed of any adhering catalyst particles, starting materials and/or products. Stripping is carried out by means of a stripping gas stream which can comprise inert gases such as nitrogen and argon, but preference is given to using a hydrogen-comprising stripping gas stream.

In a particularly preferred embodiment, the regeneration zone is arranged directly below the reaction zone and the stripping zone is arranged directly below the regeneration zone. The stripping zone preferably has not more than the same cross section perpendicular to the main flow direction of the particles as the regeneration zone and particularly preferably has a cross section which is at least 20% smaller. The regeneration zone preferably has not more than the same cross section perpendicular to the main flow direction of the particles as the reaction zone and particularly preferably has a cross section which is at least 20% smaller, with the stripping zone in turn having at most the same cross section, preferably a cross section which is at least 20% smaller, perpendicular to the main flow direction of the particles as the regeneration zone.

If the regeneration zone is located directly below the reaction zone, the starting materials for the reaction to be carried out in step (a) are preferably fed into the lower part of the reaction zone, particularly preferably in the lower third and very particularly preferably in the lowest quarter of the reaction zone. The gaseous and/or liquid products formed in the reaction are usually discharged from the reaction zone in the upper part of the reaction zone, preferably in the upper third and particularly preferably in the uppermost quarter of the reaction zone and are very particularly preferably taken off at the top, which is particularly useful when gaseous products are involved.

In regeneration of the catalyst particles by introduction of a hydrogen-comprising regeneration gas stream, this stream is, when the regeneration zone is arranged below the reaction zone, fed into the lower part, preferably the lowest third and particularly preferably the lowest quarter, of the regeneration zone in step (b2).

After regeneration of the catalyst particles, the regenerated catalyst particles are recirculated to the reaction zone. Here, the catalyst particles can be recirculated outside or within the reactor.

When the regeneration zone directly adjoins the reaction zone, preference is given, according to the invention, to the transfer of the carbonized catalyst and optionally the inert heat transfer particles from the reaction zone into the regeneration zone being carried out indirectly, i.e. without a diversion, in the region in which the two reaction zones physically adjoin. If a reaction in which methane can be used as starting material, in particular the DHAM, is carried out in step (a), the transfer of at least part of the gas stream M formed in the regeneration in step (b2) from the regeneration zone into the reaction zone is also preferably carried out directly. The average flow direction of the gas stream M is, according to the invention, counter to the average flow direction of the carbonized catalyst particles. In the case of external recirculation, the regenerated catalyst is, when the regeneration zone is arranged below the reaction zone, preferably recirculated to the upper part of the reaction zone, more preferably to the upper third and very particularly preferably the uppermost quarter of the reaction zone in step (b3). In particular, the catalyst particles are returned to the reaction zone from above.

Both in the reaction to be carried out in step (a) and the regeneration of the deactivated catalyst in a nonoxidative atmosphere as per step (b2), the catalyst particles and the inert heat transfer particles can be present as a fluidized bed or moving bed in the corresponding reactor type suitable for this purpose. According to the invention, the catalyst particles and the inert heat transfer particles are preferably present as a fluidized bed in the reaction zone, in the regeneration zone or in both zones, with particular preference being given to operating the reaction zone and the regeneration zone as a combined fluidized bed divided into zones.

According to the invention, the operating parameters, reactor configuration and reactor dimensions are preferably selected so that there is essentially no backmixing of gas from the reaction zone into the regeneration zone, even when the regeneration zone directly adjoins the reaction zone, to prevent, if possible, introduction of starting materials from the reaction zone into the regeneration zone. This could, in the case of particular reactions, have an adverse effect on the reductive regeneration of the catalyst. For example, methane is formed in the regeneration of the catalyst which has been deactivated in the DHAM of C₁-C₄-aliphatics by means of hydrogen, and introduction of these aliphatics, in particular methane, therefore has an adverse effect on the reaction equilibrium of the regeneration.

The regeneration zone is particularly preferably operated as a fluidized bed, with essentially no internal mixing of the gas phase occurring. Internal mixing should be suppressed as far as possible in order to avoid or at least reduce backmixing of the stream comprising starting materials from the reaction zone into the regeneration zone and thus ensure an atmosphere which is virtually pure reducing agent and/or inert gas in the regeneration zone. In the regeneration of the catalyst particles by means of hydrogen, a very pure hydrogen atmosphere should be provided in this way. In particular, in the case of the DHAM of C₁-C₄-aliphatics, a gas phase which is very low in methane in the regeneration zone directly adjoining the reaction zone leads to better regeneration of the catalyst particles to be regenerated.

The conditions for operating the catalyst bed comprising catalyst particles and optionally inert heat transfer particles with very low internal mixing are known to those skilled in the art. Information on the choice of parameters/operating conditions may be found, for example, in D. Kunii, O. Levenspiel “Fluidization Engineering Second Edition, Boston, Chapter 9, pages 211 to 215, and Chapter 10, pages 237 to 243.

A further possible way of reducing internal mixing in the regeneration zone is incorporation or installation of devices which prevent internal mixing. These devices can be, for example, perforated plates, structured packings, guide plates and further internals known to those skilled in the art. According to a preferred embodiment, at least one such device is arranged in the regeneration zone. The extent of internal mixing can, for example, be determined by means of the vertical dispersion coefficient.

When a DHAM is carried out in step (a), preference is given to less than 10 mol % of C₁-C₄-aliphatic, in particular methane, based on the regeneration gas stream, being introduced by backmixing into the regeneration zone from the reaction zone.

According to the invention, the reaction zone is preferably separated from the regeneration zone by at least one device which allows passage of the reaction streams and the catalyst particles and inert heat transfer particles and is arranged in the transition region between the reaction zone and the regeneration zone. These devices can be perforated plates, guide plates, structured packings and further internals which are known for this purpose to those skilled in the art, as are described, for example, in Handbook of Fluidization and Fluid-Particle Systems, New York, 2003, Editor W. Yang, Chapter 7, pages 134 to 195. The backmixing of catalyst particles and the reaction gases between these two zones can be influenced by means of these devices. For the purposes of the invention, the term reaction gases refers to the totality of the gas streams involved in the reaction zone and the regeneration zone, i.e. the gas streams E, P, any hydrogen-comprising regeneration gas stream and any stripping gas stream.

The reaction zone is preferably operated, according to the invention, as bubble-forming or turbulent fluidized bed, usually at superficial gas velocities of from 10 to 100 cm/s.

Preference is given, according to the invention, to the catalyst particles and the inert heat transfer particles on the one hand and the various streams (starting material, stream used for regenerating the catalyst particles, stripping gas) on the other hand being conveyed in countercurrent. If the regeneration zone is arranged, as per the above-described preferred embodiment, directly below the reaction zone, this means that the inert heat transfer particles flow on average from the top downward and the feed streams, product streams, streams introduced for regenerating the catalyst particles and the stripping gas streams have an average flow direction from the bottom upward. As a result of the internal circulation of solids, the catalyst particles move between the reaction zone and the regeneration zone. The particles which have been carbonized in the reaction zone move on average from the top downward while the particles which have been regenerated in the regeneration zone move on average from the bottom upward.

During the reaction in step (a), the inert heat transfer particles are present in admixture with the catalyst particles. In step (c1), the inert heat transfer particles are separated off from the catalyst particles. Here, it is important for the purposes of the invention that the separation is carried out in such a way that essentially only the inert heat transfer particles get into the heating zone, while the catalyst particles are regenerated in a nonoxidative atmosphere. The separation of the inert heat transfer particles from the catalyst particles can be carried out by various methods. Suitable methods are various processes such as segregation, classification, magnetic separation, sieving, electrostatic separation or any other possible way of separating different particles. Segregation, classification and sieving are based, for example, on different sizes and densities of the catalyst particles and the inert heat transfer particles, and magnetic separation is carried out on the basis of different magnetic properties of the particles to be separated.

According to the invention, the inert heat transfer particles are preferably separated off from the catalyst particles by segregation. For this purpose, the inert heat transfer particles and the catalyst particles have to have different fluidization properties, so that they are fluidized at different gas velocities. The general rule of thumb is that larger particles having a higher density tend to collect in the lower part in a fluidized bed, whereas smaller particles having a lower density are significantly lighter and are fluidized at lower gas flows and therefore migrate upward when the flow parameters and particle properties are chosen appropriately.

If the inert heat transfer particles are larger and have a higher density than the catalyst particles, separation of the two types of particles is achieved by passing a gas stream through the particle mixture at a flow rate which is high enough to fluidize the largest catalyst particles but is not sufficient to fluidize the inert heat transfer particles. After separation of the inert heat transfer particles and the catalyst particles, the gas stream flowing through the inert heat transfer particles can be changed so that the inert heat transfer particles are fluidized again and can be transported easily into the heating zone.

The separation of the inert heat transfer particles and the catalyst particles is ideally complete, and the heat particles which have been separated off preferably comprise not more than 0.1% by weight of catalyst particles, based on the total amount of the particles separated off (heat transfer particles separated off and catalyst particles separated off together with these).

The inert particles preferably have about twice the particle density and ten times the size.

The separation of the inert heat transfer particles from the catalyst particles in step (c1) can be carried out between steps (a) and (b), during step (b) or after step (b). The inert heat transfer particles are preferably separated off during or after step (b). The separation of the inert heat transfer particles from the catalyst particles is particularly preferably carried out in step (b2) during the regeneration of the catalyst particles in the regeneration zone. Preference is likewise given to separating off the inert heat transfer particles in the stripping zone, should such a stripping zone be present.

If the regeneration zone and any stripping zone present directly adjoin the reaction zone directly below the latter and the inert heat transfer particles are separated off from the catalyst particles in the regeneration zone or in any stripping zone present, this is, in a preferred embodiment, carried out by segregation. Here, the inert heat transfer particles and the catalyst particles have different fluidization properties and are separated from one another by the catalyst particles and the inert heat transfer particles being fluidized differently and demixing as a result of appropriate setting of the regeneration gas flow in the regeneration zone or of the stripping gas flow in the stripping zone. The hydrogen-comprising regeneration gas stream or a hydrogen-comprising stripping gas stream which can subsequently serve as regeneration gas stream is particularly useful for this purpose.

After separation of the inert heat transfer particles from the catalyst particles, the inert heat transfer particles which have been separated off are transferred into a heating zone (step (c2)).

In the heating zone, the inert heat transfer particles are heated and the heated inert heat transfer particles are subsequently recirculated to the reaction zone (step (c3)). The inert heat transfer particles are heated in the heating zone by contact with hot inert gas, contact with hot combustion offgas, direct combustion by means of a fuel such as the starting material used in the nonoxidative dehydroaromatization of C₁-C₄-aliphatics, removal of deposits on the heat transfer particles, contact with hot surfaces, action of electromagnetic waves, electrically and/or by induction. The inert heat transfer particles which have been separated off are preferably heated in step (c3) by contact with hot combustion offgas, direct combustion of at least one fuel and/or burning-off of deposits on the heat transfer particles.

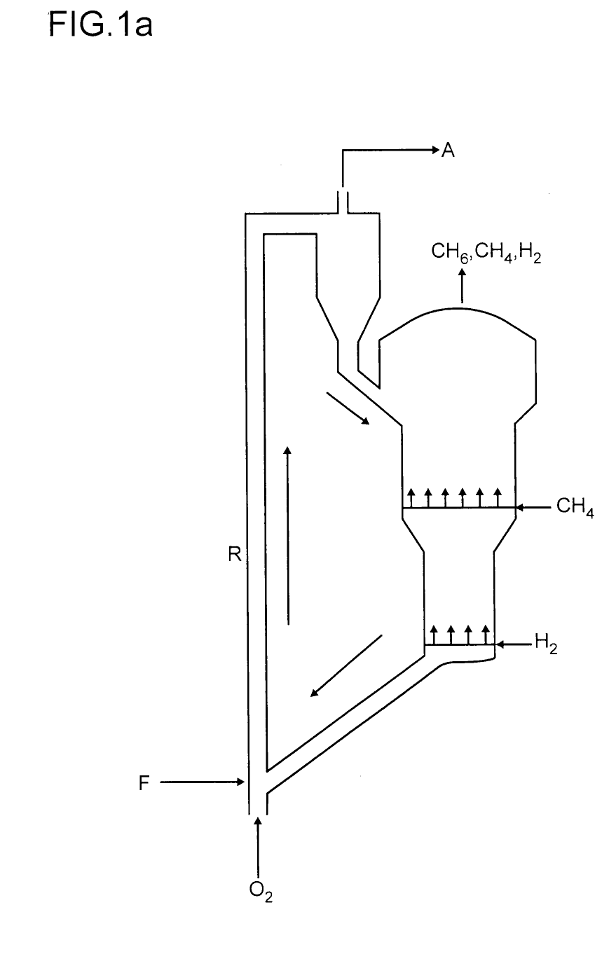

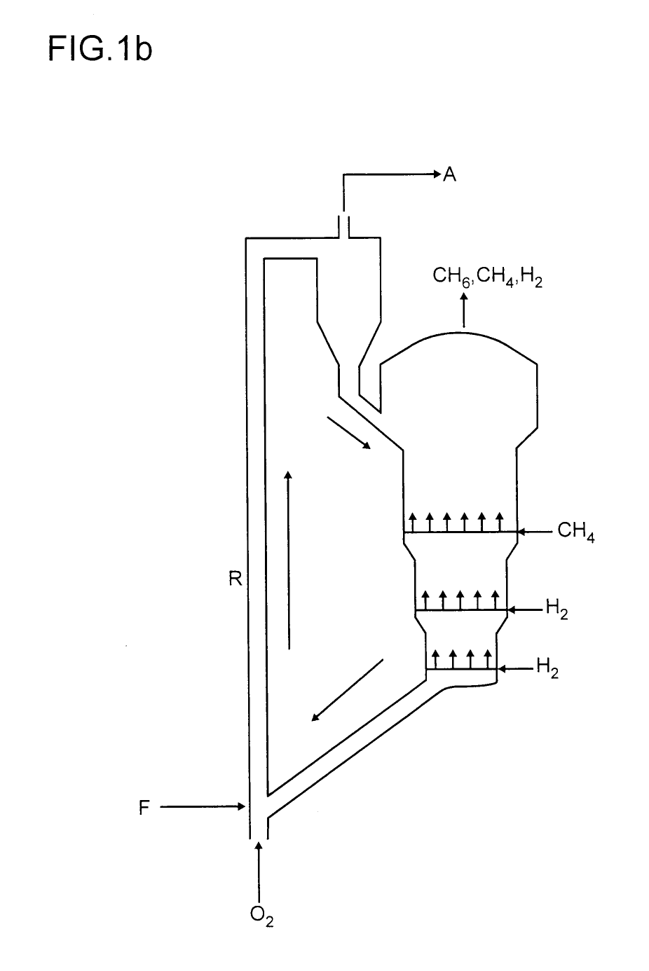

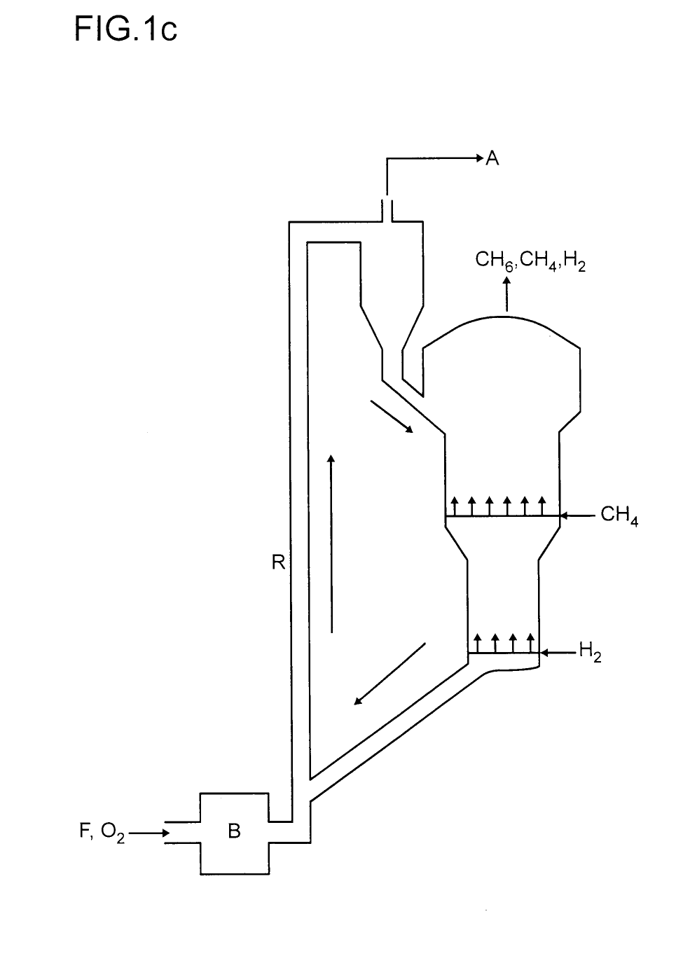

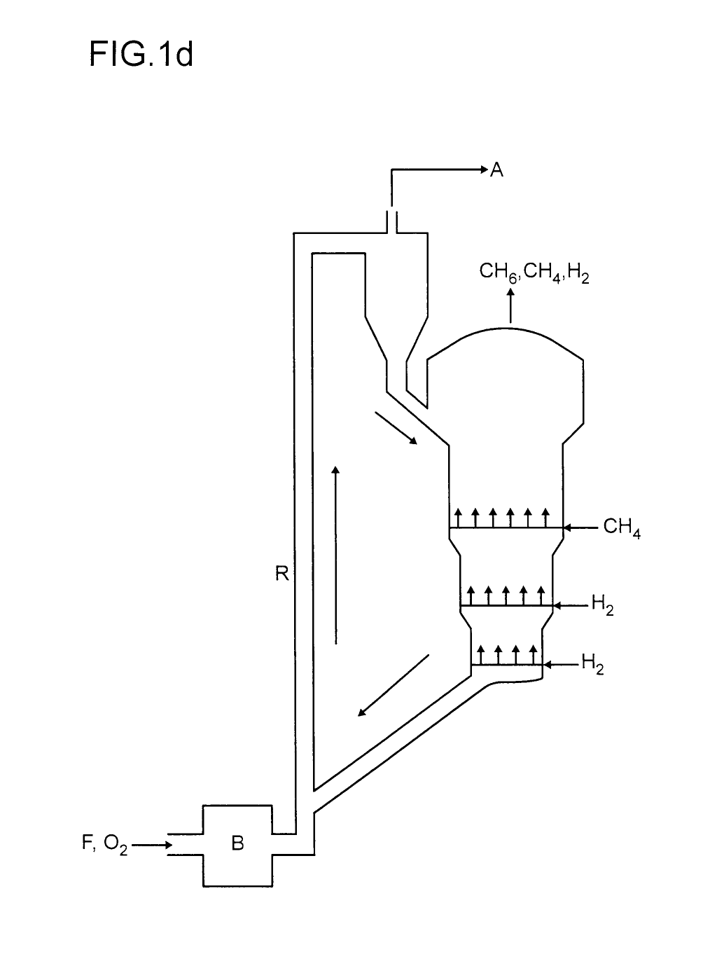

Four types of reactor which are particularly suitable for carrying out the process of the invention are illustrated in FIG. 1 for the nonoxidative aromatization of methane with regeneration of the deactivated catalyst particles by means of a hydrogen-comprising regeneration gas stream (FIGS. 1(a) to (d)).

Here, F is fuel, B is a combustion apparatus and A is the offgas formed during combustion. In the reactor schemes shown in FIGS. 1(a) and (b), the fuel (F) and oxygen, for example air, is burnt directly in the presence of the inert heat transfer particles to be heated. In the reaction schemes shown in FIGS. 1(c) and (d), the fuel (F) is burnt by means of oxygen in a burner apparatus (B) and the hot combustion offgases formed are passed over the inert heat transfer particles.

The preferred embodiment of the invention in which the regeneration zone is located directly below the reaction zone is shown in FIGS. 1(a) to (d). In each case, CH₄ is introduced into the bottom part of the reaction zone, and hydrogen is in each case introduced into the bottom part of the regeneration zone. In the embodiments shown in FIGS. 1(b) and (d), a stripping zone is located directly below the regeneration zone, with a hydrogen-comprising gas mixture being used as stripping gas here. In the embodiments shown in figures (a) and (c), the inert heat transfer particles are separated from the catalyst particles in the regeneration zone and transferred via the downward-conducting pipe into a riser (R) where they are heated. The heat transfer particles are returned in an upward direction to the reaction zone. In the embodiments shown in FIGS. 1(b) and (d), the inert heat transfer particles are separated off from the catalyst particles in the stripping zone and heated in the same way as described above in the riser (R), conveyed in an upward direction and returned from above to the reaction zone.

The invention is illustrated below with the aid of examples.

### EXAMPLE 1

### Influence of Water Vapor in the Reaction Gas of the DHAM

About 1.6 g of the catalyst (6% of Mo, 1% of Ni on an H-ZSM-5 support having an SiO₂:Al₂O₃ ratio of 25) were heated to 500° C. under a helium atmosphere in the reactor tube (internal diameter=4 mm). At this temperature, methane was introduced and the catalyst was maintained at this temperature for 30 minutes before being brought to the reaction temperature of 700° C. under methane comprising 10% by volume of helium. The catalyst was then operated for about 35 hours at 700° C., 1 bar, 10% by volume of He in methane and a GHSV of 500 h⁻¹. During the reaction, the feed gas was passed through a saturator for 180 minutes and 2.8% by volume of water vapor was in this way added to the feed gas mixture. After the reaction, the degree of crystallinity of the ZSM-5 zeolite support was determined by means of X-ray diffraction (XRD) on the catalysts which had been removed from the reactor.

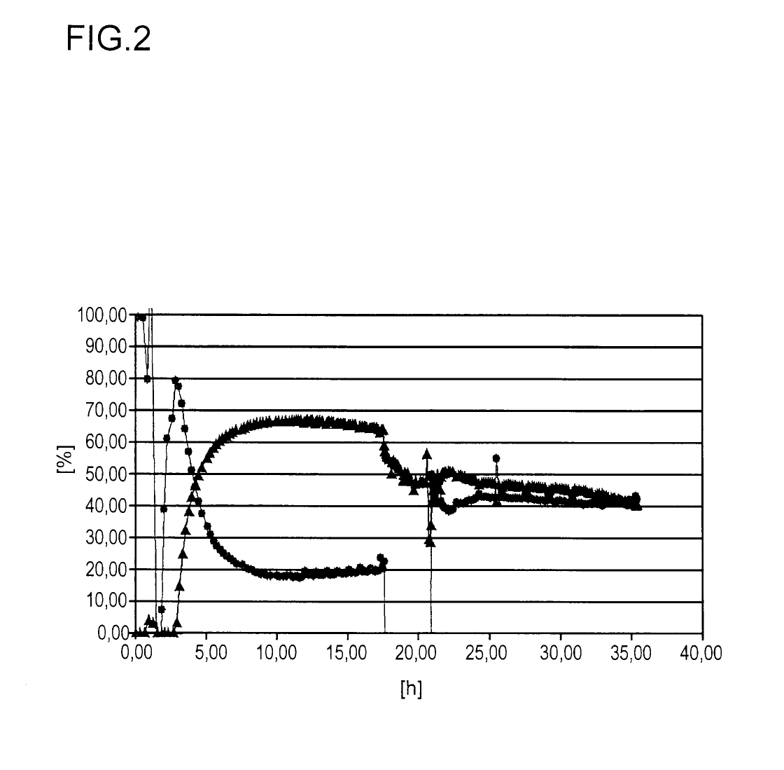

The benzene selectivities and the measured degrees of crystallinity are shown in Table 1. In Table 1, the time 0 min corresponds to the commencement of introduction of water vapor, which was started 17.5 hours after the beginning of the reaction. The benzene selectivity S_{B} (solid triangles) and the selectivity for carbonaceous material (solid circles) as a function of the reaction time t are shown in FIG. 2.

TABLE 1

| | | Degree of crystallinity of | |
|---|---|---|---|
| Reaction time t | Time of introduction | ZSM-5 in the catalyst | |
| [h] | of water vapor | removed from the reactor | S_{B} |
| 17.5 | 0 min | 71% | 68% |
| 18 | 30 min | 68% | 66% |
| 18.7 | 40 min | 67% | 64% |
| 20.5 | 180 min | 54% | 51% |

S_{B}: Benzene selectivity, amount of methane converted into benzene based on the amount of methane reacted

The results of the X-ray diffraction show that the presence of water vapor in the reaction gas damages the zeolite support under the reaction conditions, leading to an irreversible reduction in the benzene selectivity over the catalyst. The longer the catalyst is exposed to water, the more does the benzene selectivity of the catalyst decrease. In a continuous process, the presence of water vapor in the reaction gas must therefore be avoided.

### EXAMPLE 2

Example 2 was carried out using a glass tube having an internal diameter of 40 mm and a total height of about 2.5 m. A glass frit was located at the bottom of the plant and gas was introduced and distributed via this. A pipe running obliquely downward was installed at the side at the lower end of the plant to allow a sample of solid to be taken. Nitrogen was used as fluidizing gas. To minimize electrostatic effects during the experiments, the nitrogen was passed through a wash bottle at room temperature in order to humidify it with a little water.

As model substance for the catalyst particles, use was made of an aluminum oxide powder (Puralox SCCa 57/170, from Sasol) which had been impregnated with an aqueous sodium chloride solution having a concentration of 6 mol/l. The amount of sodium chloride solution corresponded to about 40% by weight of the amount of particles.

The aluminum oxide content of the particle samples discharged was determined by means of conductivity measurements. For this purpose, 100 ml of deionized water were added to 10 g of the sample of solid and this mixture was then stirred for about 2 minutes. The inert particles settle immediately on the bottom, and the aluminum oxide remained suspended. The conductivity was measured in the water above the particles after the latter had settled virtually completely.

### EXAMPLE 2a

### Conductivity of the Pure Materials

| | | |
|---|---|---|
| Glass spheres, washed and dried (10 g in 100 ml | 6-8 | μS/cm |
| of water): | | |
| Freshly impregnated Puralox (1 g in 100 ml of | about 1700 | μS/cm |
| water) | | |
| Unimpregnated Puralox (1 g or 10 g in | 1 | μS/cm |
| 100 ml of water) | | |
| Pure deionized water | 4 | μS/cm |
| Magnesium silicate: | 13 | μS/cm |

### EXAMPLE 2b

### Determination of the Separation Properties

The separation properties were determined using various inert particles. These were steatite particles having two different size ranges and glass spheres. The properties of the inert particles used and the catalyst particles are summarized in Table 2. The particle size distribution was in each case determined by sieve analysis.

TABLE 2

| | Inert particles 1 | Inert particles 2 | Inert particles 3 | Catalyst |
|---|---|---|---|---|
| Material | Steatite | Glass | Steatite | Al₂O₃ |
| d_{p,50} [μm] | 240 | 500 | 750 | 50 |
| ρ [kg/m³] | 1422 | 1438 | 1563 | 800 |
| u_{mf} [cm/s] | 14.3 | 28 | 41.1 | 0.3 |
| u₀ [cm/s] | 10 | 20 | 30 | |

d_{p,50}: average particle diameter
ρ: bulk density
u_{mf}: minimum fluidization velocity
u₀: superficial gas velocity set in the experiment

The output of catalyst particles was determined by initially charging the aluminum oxide particles as model substance (bed height about 350 mm) and fluidizing them by means of nitrogen at the superficial gas velocities indicated in Table 2. The inert particles were subsequently introduced continuously from above via a metering screw having a metering rate of 120 g/min. After inert particles had accumulated in the lower region of the fluidized bed, the same mass flow of inert particles as was introduced at the top was taken off continuously from this lower region. After the plant had been operating in a steady state for about 5 minutes, a sample was taken from the stream taken off and analyzed by the method described above.

The results for the three inert particles examined are shown in table 3.

TABLE 3

| | Inert particles 1 | Inert particles 2 | Inert particles 3 |
|---|---|---|---|
| u_{mf,in}/u_{mf,cat} | 48 | 93 | 137 |
| m_{cat} | 0.33% | 0.04% | 0% |

u_{mf,in} in: minimum fluidization velocity for the inert particles
u_{mf,cat} cat: minimum fluidization velocity for the catalyst particles
m_{cat}: mass of catalyst particles based on the total mass of the particle sample taken off, in %

1. A process for carrying out endothermic, heterogeneously catalyzed reactions, which comprises the steps
(a) carrying out the reaction in a reaction zone in the presence of a mixture comprising catalyst particles and inert heat transfer particles,
(b) regeneration of the catalyst particles, comprising
(b1) transfer of the mixture comprising catalyst particles and optionally inert heat transfer particles into a regeneration zone,
(b2) regeneration of the catalyst particles and optionally the inert heat transfer particles in a nonoxidative atmosphere and
(b3) recirculation of the regenerated catalyst particles to the reaction zone and
(c) introduction of heat into the reaction zone, which comprises the steps
(c1) separation of the inert heat transfer particles from the catalyst particles between step (a) and (b), during step (b) or after step (b),
(c2) transfer of the inert heat transfer particles which have been separated off into a heating zone and
(c3) heating of the inert heat transfer particles and recirculation of the heated inert heat transfer particles to the reaction zone.

2. The process according to claim 1, wherein the catalyst particles comprise zeolite.

3. The process according to claim 1 or 2, wherein the reaction in step (a) is the nonoxidative dehydroaromatization of C₁-C₄-aliphatics.

4. The process according to claim 1 or 2, wherein the regeneration is carried out by introduction of a hydrogen-comprising regeneration gas stream.

5. The process according to claim 1 or 2, wherein the inert heat transfer particles are separated off by segregation, classification, magnetic separation, electrostatic separation and/or sieving.

6. The process according to claim 1 or 2, wherein the heat transfer particles are separated off during or after step (b).

7. The process according to claim 1 or 2, wherein catalyst particles are present in the reaction zone as a moving bed or fluidized bed.

8. The process according to claim 1, wherein the regeneration zone for regenerating the catalyst particles directly adjoins the reaction zone.

9. The process according to claim 8, wherein the reaction zone and the regeneration zone are operated as a combined fluidized bed divided into zones.

10. The process according to claim 8 or 9, wherein the regeneration zone is arranged below the reaction zone and has at most the same cross section perpendicular to the main flow direction of the particles as the reaction zone.

11. The process according to claim 8, wherein a stripping zone adjoins the regeneration zone.

12. The process according to claim 11, wherein the stripping in the stripping zone is carried out by introduction of a hydrogen-comprising stripping gas stream.

13. The process according to claim 11 or 12, wherein the stripping zone is arranged below the regeneration zone and has at most the same cross section perpendicular to the main flow direction of the particles as the regeneration zone.

14. The process according to claim 8, wherein the heat transfer particles are separated off from the catalyst particles in the regeneration zone.

15. The process according to claim 11, wherein the heat transfer particles are separated off from the catalyst particles in the stripping zone.

16. The process according to claim 14 or 15, wherein the inert heat transfer particles and the catalyst particles have different fluidization properties and are separated from one another by the catalyst particles and the inert heat transfer particles being fluidized to differing extents and demixing as a result of appropriate setting of the regeneration gas flow in the regeneration zone or of the stripping gas flow in the stripping zone.

17. The process according to claim 1 or 2, wherein the inert heat transfer particles which have been separated off are heated in step (c3) by contact with hot inert gas, contact with hot combustion offgas, direct combustion of at least one fuel, burning-off of deposits on the heat transfer particles, contact with hot surfaces, action of electromagnetic waves, electrically and/or by induction.

18. The process according to claim 1 or 2, wherein the inert heat transfer particles which have been separated off are heated in step (c3) by contact with hot combustion offgas, direct combustion of at least one fuel and/or burning-off of deposits on the heat transfer particles.

19. The process according to claim 1 or 2, wherein the heat transfer particles which have been separated off comprise not more than 0.1% by weight of catalyst particles, based on the total amount of the particles which have been separated off.

20. The process according to claim 1 or 2, wherein the weight ratio of heat transfer particles to catalyst particles in the reaction zone is from 2:1 to 1:10.

21. The process according to claim 1 or 2, wherein the inert heat transfer particles are selected from the group consisting of glass spheres, ceramic spheres, silicon carbide particles, Al₂O₃ particles, steatite particles and sand.
